(19) United States
(12) Patent Application Publication
(10) Pub. No.: US 2026/0297053 A1
(43) Pub. Date: Oct. 1, 2026

(54) METHOD FOR PREPARING BISAMIDE INSECTICIDE AND INTERMEDIATE THEREOF

(71) Applicant: LIER CHEMICAL CO., LTD., Mianyang (CN)

(72) Inventors: Weiping Liang, Chengdu (CN); Mingxue Wu, Chengdu (CN); Jun He, Chengdu (CN); Ke Cheng, Chengdu (CN)

(21) Appl. No.: 19/475,610

(22) PCT Filed: Apr. 1, 2024
(86) PCT No.: PCT/CN2024/085205

§ 371 Date: Oct. 15, 2025

(30) Foreign Application Priority Data
Apr. 17, 2023 (CN) 202310407854.0

(51) Int. Cl. C07D 401/04

(52) U.S. Cl. CPC C07D 401/04

(57) ABSTRACT

The present invention relates to a method for preparing a bisamide insecticide or an intermediate thereof. Specifically, the present invention relates to a method for preparing a compound of Formula I. The method comprises the step of reacting a carboxylic acid as represented by Formula I-1 with a halogenating agent and elemental halogen. The method can substantially improve product yield, producing relatively high economic benefits.

The present application is based on and claims priority to the Chinese application with application number CN 202310407854.0 and application date Apr. 17, 2023, the content of which is hereby incorporated by reference in its entirety.

### TECHNICAL FIELD

The present invention relates to the field of organic synthesis, and specifically to a method for preparing a bisamide insecticide and intermediate thereof.

### BACKGROUND ART

3-Halogenated-1-(3-chloro-2-pyridyl)-1H-pyrazole-5-benzoic acid and derivatives thereof (with structural formula as shown in Formula I) are important intermediates for the preparation of ortho-amido benzamide insecticides such as chlorantraniliprole, cyantraniliprole, tetrachlorantraniliprole, cyclaniliprole, tetraniliprole, tiorantraniliprole, Fuyangchongxian'an (ZJ3757) and Saichongxian'an (JS9117).

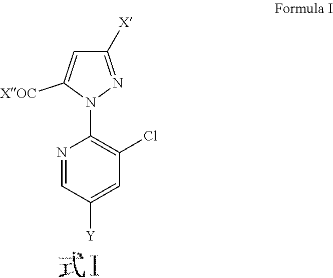

It is disclosed in CN101550130B that a carboxylic acid is subjected to acyl halogenation and oxidation reaction simultaneously under the action of acyl halogenating agents such as oxalyl chloride, thionyl chloride, phosphorus tribromide, phosphorus trichloride, phosphorus pentabromide or phosphorus pentachloride to obtain the target acyl halide. The method uses toluene and hexane as solvents with relatively good effects, but the yield is not ideal (86%), and the solubility of carboxylic acid in toluene or hexane is very poor.

### CONTENTS OF THE PRESENT INVENTION

In order to solve the problems existing in the prior art, the present invention provides a novel method for preparing acyl halide. The method of the present invention can be used to obtain a target product with high-purity and high yield, which can be directly used in subsequent reactions. The method is particularly suitable for the preparation of bisamide insecticides or intermediates thereof, and can bring higher economic benefits to the production of agricultural chemicals.

Specifically, the present invention provides a method for preparing a compound represented by Formula I, comprising the following steps:
reacting a carboxylic acid represented by Formula I-1 with a halogenating agent and an elemental halogen to obtain the compound represented by Formula I:

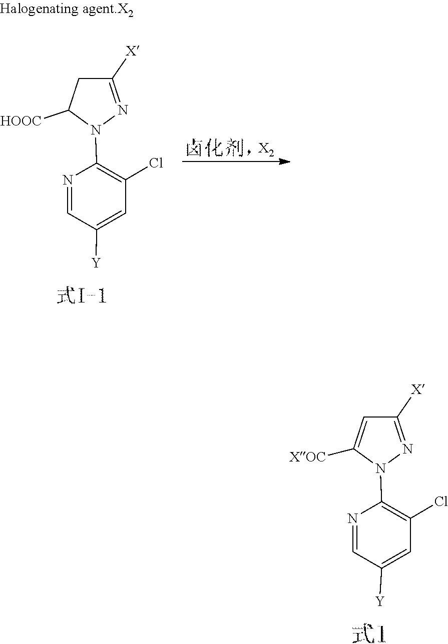

in Formula I and Formula I-1, X is halogen; X′ is halogen, C₁₋₆ alkyl, C₁₋₆ alkoxy or C₁₋₆ haloalkoxy; X″ is halogen; Y is H or halogen; X, X′ and X″ are the same or different.

In some embodiments, X is Cl or Br; X′ is Cl, Br, C₁₋₂ alkyl, C₁₋₂ alkoxy or C₁₋₂ haloalkoxy; X″ is Cl or Br; Y is H, Cl or Br; X, X′ and X″ are the same or different.

In some embodiments, X is Cl; X′ is Cl or Br; X″ is Cl or Br; Y is H or Cl; X, X′ and X″ are the same or different.

In some embodiments, X is Cl; X′ is Br; X″ is Cl; Y is H.

In some embodiments, the halogenating agent is a chlorinating agent or a brominating agent.

In some embodiments, the chlorinating agent is selected from the group consisting of sulfinyl chlorides (e.g., thionyl chloride), sulfonyl chlorides (e.g., methanesulfonyl chloride or trichloromethanesulfonyl chloride), acyl chlorides (e.g., methoxyacetyl chloride, oxalyl chloride, phosgene or triphosgene) and phosphorus chlorides (e.g., phosphorus trichloride or phosphorus pentachloride).

In some embodiments, the brominating agent is selected from the group consisting of thionyl bromide and phosphorus tribromide.

In some embodiments, the molar ratio of the carboxylic acid to the halogenating agent (for a polymer such as triphosgene, the calculation is based on its monomer unit) is 1:(1.0-3.0), preferably 1:(1.0-2.0). When the halogenating agent is a polymer, such as triphosgene, the molar ratio of the carboxylic acid to the halogenating agent refers to the molar ratio of the carboxylic acid to all monomers that can be provided by the polymer for the reaction.

In some embodiments, the elemental halogen is chlorine or bromine.

In some embodiments, the halogenating agent is thionyl chloride, and the elemental halogen is chlorine.

In some embodiments, the reaction is carried out in a solvent.

In some embodiments, the solvent is one or more selected from the group consisting of acetonitrile, chlorobenzene, dioxane, tetrahydrofuran, dichloromethane, dichloroethane, chloroform and carbon tetrachloride.

In some embodiments, the solvent is acetonitrile.

In some embodiments, the method comprises the following steps:
adding the carboxylic acid, the halogenating agent (e.g., thionyl chloride) and the elemental halogen (e.g., chlorine) to the solvent, respectively, to obtain a solution containing the carboxylic acid, a solution containing the halogenating agent and a solution containing the elemental halogen;
adding the solution containing the carboxylic acid and the solution containing the halogenating agent obtained above to the solution containing the elemental halogen, and reacting to obtain the compound represented by Formula I.

In some embodiments, the solution containing the carboxylic acid and the solution containing the halogenating agent are added to the solution containing the elemental halogen simultaneously. In some embodiments, the term “simultaneously” refers to that the operations of adding the solution containing the carboxylic acid and adding the solution containing the halogenating agent overlap partially or completely in time.

In some embodiments, the temperature of the reaction system during the addition process is controlled. In some embodiments, the temperature of the reaction system during the addition process is controlled to be 5 to 70° C., for example, 5 to 45° C., 5 to 40° C., 5 to 30° C., 5 to 20° C., or 10 to 15° C.

In some embodiments, the reaction is carried out at −10 to 80° C., preferably 10 to 50° C., more preferably 10 to 45° C., for example, 10 to 40° C., 15 to 45° C. or 30 to 45° C.

In some embodiments, the carboxylic acid reacts with the halogenating agent and the elemental halogen in the presence of an acid binding agent.

In some embodiments, the acid binding agent is one or more selected from the group consisting of triethylamine, pyridine, 3-methylpyridine and 2-methylpyridine. In some embodiments, the acid binding agent is triethylamine. In some embodiments, the acid binding agent is pyridine. In some embodiments, the acid-binding agent is 3-methylpyridine.

In some embodiments, the carboxylic acid reacts with the halogenating agent and the elemental halogen in the presence of a catalyst.

In some embodiments, the catalyst is one or more selected from the group consisting of N,N-dimethylformamide, N,N-dimethylaniline, pyridine and triethylamine. In some embodiments, the catalyst is selected from N,N-dimethylformamide.

In some embodiments, the carboxylic acid reacts with the halogenating agent and the elemental halogen in the presence of an acid-binding agent and a catalyst.

In some embodiments, the carboxylic acid reacts with the halogenating agent and the elemental halogen in the presence of one or more of the following substances: triethylamine, pyridine, 3-methylpyridine, 2-methylpyridine, N,N-dimethylformamide and N,N-dimethylaniline. In some embodiments, the carboxylic acid reacts with the halogenating agent and the elemental halogen in the presence of N,N-dimethylformamide. In some embodiments, the carboxylic acid reacts with the halogenating agent and the elemental halogen in the presence of 3-methylpyridine. In some embodiments, the carboxylic acid reacts with the halogenating agent and the elemental halogen in the presence of pyridine. In some embodiments, the carboxylic acid reacts with the halogenating agent and the elemental halogen in the presence of triethylamine and N,N-dimethylformamide. In some embodiments, the carboxylic acid reacts with the halogenating agent and the elemental halogen in the presence of 3-methylpyridine and N,N-dimethylformamide.

In some embodiments, the molar ratio of the acid-binding agent to the carboxylic acid is 0.05 to 0.5, such as 0.05 to 0.2, and for another example, 0.1 to 0.2.

In some embodiments, the molar ratio of the catalyst to the carboxylic acid is 0.05 to 0.5.

By using the above method of the present invention, the yield of the target product is above 80%, for example, above 85%, above 90%, above 91%, above 92%, above 93%, above 94%, above 95%, above 96%, above 97%, above 98%, above 99%. Since the compound represented by Formula I prepared by the present invention has high activity, for the convenience of calculation, in some cases, the yield of the compound represented by Formula I is calculated based on the carboxylic acid obtained by hydrolysis of the compound represented by Formula I. The purity of the corresponding carboxylic acid obtained is above 80%, for example, above 85%, above 90%, above 91%, above 92%, above 93%, above 94%, above 95%, above 96%, above 97%, above 98%, above 99%.

In another aspect, the present invention provides a method for preparing a compound represented by Formula II, which comprises the following steps:
using the Formula I-1 as a raw material, and performing the operation according to any one of the methods as described above to obtain the compound represented by Formula I;
hydrolyzing the compound represented by Formula I to obtain the compound represented by Formula II,

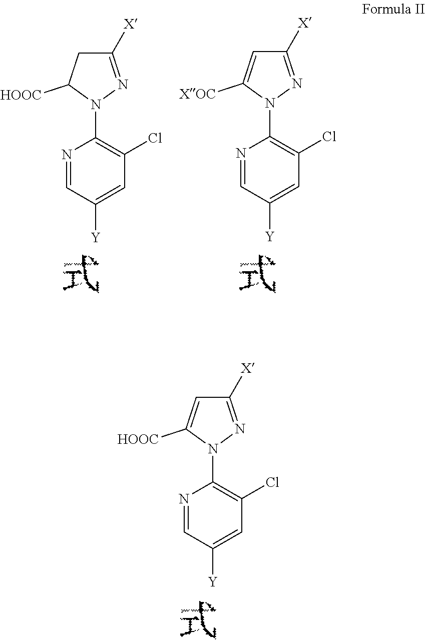

wherein, the groups in Formula I, I-1 and II are as defined above.

The compound represented by Formula I as prepared above can be directly used in the next reaction, such as amidation reaction, after removing the elemental halogen and low-boiling impurities in the solvent by vacuum distillation. In other embodiments, the compound represented by Formula I as prepared above can also be added to water (preferably adjusted to pH 2 to 3 with, for example, NaOH aqueous solution) and hydrolyzed to form a corresponding acid (e.g., the carboxylic acid represented by Formula II herein), which is then purified by extraction or crystallization to obtain a pure product of the acid, and the acid then reacts with an amino fragment to perform the next step such as amidation reaction. Therefore, the compound represented by Formula I as prepared by the present invention can be used as an active intermediate for the preparation of various key intermediates of amide bond-containing chemicals, such as bisamide pesticides.

Therefore, in another aspect, the present invention provides a method for preparing a compound represented by Formula III, which comprises the following steps:
a carboxylic acid represented by Formula I-1 is reacted with a halogenating agent and a elemental halogen according to the method described in any one of the first aspects;
the product obtained from the previous step is reacted with a compound represented by Formula IV to obtain a compound represented by Formula III; preferably, the reaction is carried out in the presence of an acid-binding agent (e.g., 3-methylpyridine); preferably, the reaction is carried out at 10 to 60° C. (e.g., 10 to 45° C., 10 to 45° C., 10 to 30° C., 15 to 25° C., or 20 to 25° C.);

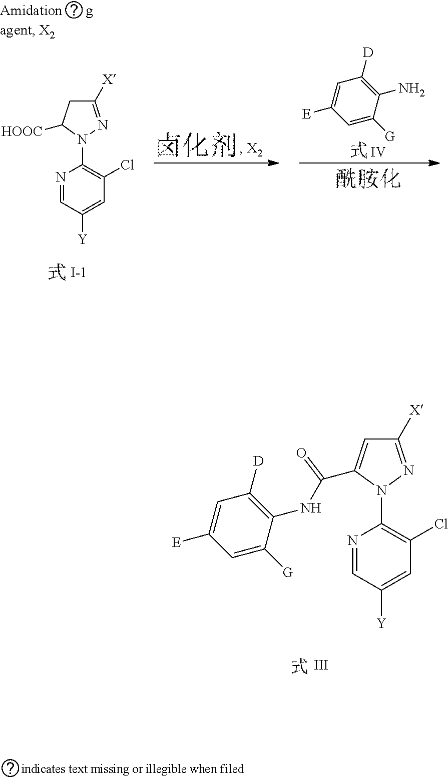

wherein, D is halogen or C₁₋₆ alkyl; E is halogen or cyano; G is —C(O)NH—C₁₋₆ alkyl, —C(S)NH—C₁₋₆ alkyl, —C(O)NH—C₁₋₆ alkylene-C₃₋₆ cycloalkyl, —C(S)NH—C₁₋₆ alkylene-C₃₋₆ cycloalkyl, or 5- to 6-membered heteroaryl unsubstituted or substituted with halogen or C₁₋₆ alkyl; the remaining groups are as defined above.

In some embodiments, D is Cl, Br or C₁₋₂ alkyl; Eis Cl, Br or cyano; G is —C(O)NH—C₁₋₄ alkyl, —C(S)NH—C₁₋₄ alkyl, —C(O)NH—C₁₋₂ alkylene-C₃₋₆ cycloalkyl, —C(S)NH—C₁₋₂ alkylene-C₃₋₆ cycloalkyl, or 5-membered heteroaryl unsubstituted or substituted with halogen or C₁₋₂ alkyl.

In some embodiments, D is Cl, Br or methyl; E is Cl or cyano; G is selected from

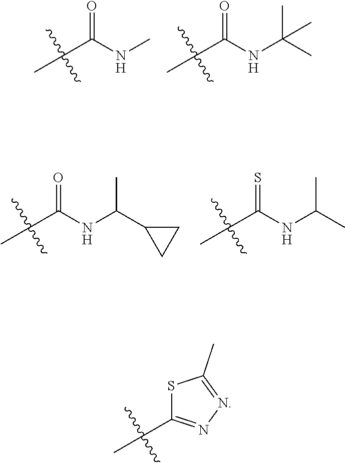

The conditions for amidation reaction described herein are known in the art and are not particularly limited here. For details, please refer to the teachings in textbooks or literature.

In some embodiments, the compound represented by Formula III is a bisamide insecticide.

In some embodiments, the compound represented by Formula III is an ortho-amido benzamide insecticide.

In some embodiments, the ortho-amido benzamide insecticide is selected from the group consisting of chlorantraniliprole, cyantraniliprole, tetrachlorantraniliprole, cyclaniliprole, tetraniliprole, tiorantraniliprole, Fuyangchongxian'an (ZJ3757) and Saichongxian'an (JS9117).

In another aspect, the present invention provides a use of the method as described in any one of the above aspects in the manufacture of a bisamide insecticide.

In some embodiments, the bisamide insecticide is an ortho-amido benzamide insecticide.

In some embodiments, the ortho-amido benzamide insecticide is selected from the group consisting of chlorantraniliprole, cyantraniliprole, tetrachlorantraniliprole, cyclaniliprole, tetraniliprole, tiorantraniliprole, Fuyangchongxian'an (ZJ3757) and Saichongxian'an (JS9117).

### Definition of Terms

As used herein, unless otherwise specified, the scientific and technical terms used have the meanings commonly understood by those skilled in the art. At the same time, in order to better understand the present invention, the definitions and explanations of the relevant terms are provided below.

As used herein, the singular forms “a”, “an” and “the” are also intended to include the plural forms unless the context clearly indicates otherwise. In addition, the terms “include”, “comprise”, “have”, “contain” or their variations are open-ended, and not exclusive or exhaustive. Furthermore, the terms “herein”, “preamble” and “hereinafter” or words of similar meaning shall refer to the entire application rather than any particular part thereof.

The term “halogen” is defined to include F, Cl, Br and I.

The term “alkyl” is defined to be a straight-chain or branched saturated aliphatic hydrocarbon group. For example, the term “C₁₋₆ alkyl” refers to an alkyl of 1 to 6 carbon atoms, and for further example, C₁₋₅ alkyl, C₁-4 alkyl, C₁-3 alkyl or C₁₋₂ alkyl. Common examples of alkyl include (but are not limited to) methyl, ethyl, n-propyl, isopropyl, n-butyl, isobutyl, sec-butyl, tert-butyl, n-pentyl and n-hexyl. When one hydrogen atom is further removed from the alkyl, the resulting group is the “alkylene” as described herein, such as C₁₋₆ alkylene, C₁₋₅ alkylene, C₁₋₄ alkylene, C₁₋₃ alkylene or C₁₋₂ alkylene.

The term “alkoxy” refers to a group having an “alkyl-O—” structure, wherein the alkyl is defined as described above. For example, C₁₋₆ alkoxy, C₁₋₅ alkoxy, C₁₋₄ alkoxy, C₁₋₃ alkoxy or C₁₋₂ alkoxy, etc. Common examples of alkoxy include (but are not limited to) methoxy, ethoxy, n-propoxy, isopropoxy, n-butoxy, isobutoxy, tert-butoxy, pentyloxy, hexyloxy, etc.

The term “haloalkyl” refers to an alkyl substituted with one or more (e.g., 1, 2 or 3) same or different halogen atoms, wherein the alkyl is defined as described above. For example, the term “C₁₋₆ haloalkyl” refers to a haloalkyl having 1 to 6 carbon atoms. Common examples of haloalkyl include (but are not limited to) —CH₂F, —CHF₂, —CF₃, —CH₂CF₃, —CF₂CF₃, —CH₂CH₂CF₃, —CH₂Cl, etc.

The term “haloalkoxy” refers to an alkoxy substituted with one or more (e.g., 1, 2 or 3) same or different halogen atoms, wherein the alkoxy is defined as above. For example, the term “C₁₋₆ haloalkoxy” refers to a haloalkoxy having 1 to 6 carbon atoms. Common examples of haloalkoxy include (but are not limited to) —OCH₂F, —OCHF₂, —OCF₃, —OCH₂CF₃, —OCF₂CF₃, —OCH₂CH₂CF₃, —OCH₂Cl, etc.

The term “cycloalkyl” refers to a saturated cyclic hydrocarbon group. For example, the term “C₃₋₆ cycloalkyl” refers to a cycloalkyl having 3 to 6 ring carbon atoms. Common examples of cycloalkyl include (but are not limited to) cyclopropyl, cyclobutyl, cyclopentyl, cyclohexyl.

The term “heteroaryl” refers to an aromatic ring group containing at least one ring member selected from the group consisting of N, O and S. For example, 5- to 6-membered heteroaryl, 5- to 6-membered nitrogen-containing heteroaryl, 5- to 6-membered oxygen-containing heteroaryl, etc. Common examples of heteroaryl include (but are not limited to) furanyl, thienyl, pyrrolyl, thiazolyl, isothiazolyl, thiadiazolyl, oxazolyl, isoxazolyl, oxadiazolyl, imidazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, pyridyl, pyrimidinyl, pyridazinyl, pyrazinyl, 1,2,3-triazinyl, 1,3,5-triazinyl, 1,2,4,5-tetrazinyl, etc.

The term “substitution” refers to that one or more (e.g., one, two, three, or four) hydrogen atoms are substituted with a designated atom. Unless otherwise specified, the point of attachment of the substituent may be from any suitable position of the substituted structure.

The term “one or more” refers to one or more than one under reasonable conditions, such as 2, 3, 4, 5 or more.

### DETAILED DESCRIPTION OF EMBODIMENTS

The embodiments of the present invention will be described in detail below in conjunction with examples, but those skilled in the art will understand that the following examples are only used to illustrate the present invention and should not be regarded as limiting the scope of the present invention. If no specific conditions were specified in the examples, they were carried out according to conventional conditions or conditions recommended by the manufacturer. The reagents or instruments used without indicating the manufacturer were all conventional products that can be obtained commercially.

### Example 1: Preparation of Raw Material KA-1

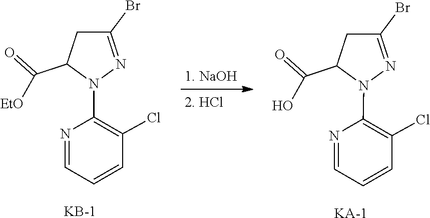

7.2 g of sodium hydroxide and 250 g of water were added to a conical flask to prepare an alkaline solution. 50 g (0.150 mol) of KB-1 was added to a three-necked flask, followed by an addition of 50 g of ethanol, the obtained mixture was stirred at 10° C., and slowly added dropwise the alkaline solution prepared above. After HPLC detection indicated the disappearance of the raw material, dilute hydrochloric acid was added to adjust the pH to 1 to 2, and then ethyl acetate was added for extraction twice, the organic phases were combined, and 45.7 g of viscous liquid was obtained by rotary evaporation, with a yield of 95.1% and an HPLC purity of 97.5%.

### Example 2

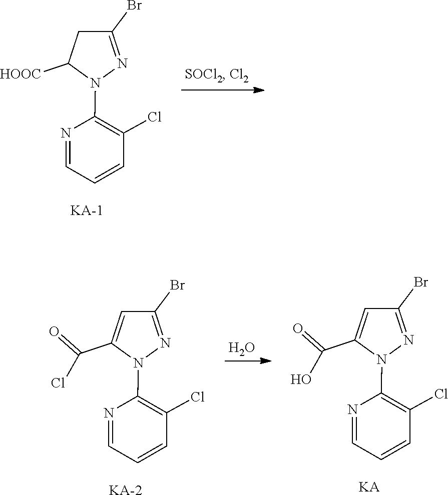

25.0 g (0.0821 mol) of KA-1, 0.830 g (0.00821 mol) of triethylamine, and 100 g of acetonitrile were added to a single-necked flask to prepare a mixed solution for later use.

14.7 g of thionyl chloride, 50.0 g of acetonitrile and 0.3 g of N,N-dimethylformamide were added to a four-necked flask, stirred at 45° C., purged with nitrogen gas for about 5 min, then purged with chlorine gas, slowly added with the prepared mixed solution using a propel pump, and the reaction was maintained at 45° C. After HPLC detection indicated the disappearance of the raw material, the reaction was terminated. 20% sodium hydroxide solution was added to adjust the pH to 2 to 3, the phases were separated, and then ethyl acetate was added for extraction. The organic phases were combined and rotary evaporated to obtain 23.9 g of product KA, with a yield of 96.2% and an HPLC purity of 96.9%.

### Example 3-1

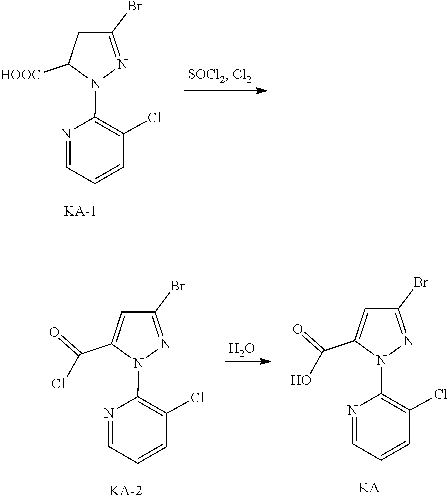

25.0 g (0.0821 mol) of KA-1 and 125 g of acetonitrile were added to a single-necked flask No. 1 to prepare a solution for later use. 14.7 g (0.123 mol) of thionyl chloride and 50 g of acetonitrile were added to a single-necked flask No. 2 to prepare a solution for later use.

First, nitrogen gas was introduced into a four-necked flask with 25 g of acetonitrile for about 5 minutes, then chlorine gas was introduced, stirred at 15° C., then added with the KA-1 solution prepared in the single-necked flask No. 1 using a propel pump No. 1, and added with the thionyl chloride solution prepared in the single-necked flask No. 2 using a propel pump No. 2. After the introduction of KA-1 and thionyl chloride was completed, the reaction system was heated to 30° C. and stirred. After HPLC detection indicated the disappearance of the raw materials, the reaction was terminated. 20% sodium hydroxide solution was added to adjust the pH to 2 to 3, the layers were separated, and then ethyl acetate was added for extraction. The organic layers were combined and rotary evaporated to obtain 23.8 g of product KA, with a yield of 96.1% and an HPLC purity of 95.6%.

### Example 3-2

According to the method of Example 3-1, after the introduction of KA-1 and thionyl chloride was completed, the reaction was carried out at 10° C. to obtain 23.5 g of product KA, with a yield of 94.8% and an HPLC purity of 94.6%.

### Example 3-3

According to the method of Example 3-1, after the introduction of KA-1 and thionyl chloride was completed, the reaction was carried out at 50° C. to obtain 23.2 g of product KA, with a yield of 93.5% and an HPLC purity of 94.1%.

### Example 3-4

25.0 g (0.0821 mol) of KA-1, 1.5 g (0.0164 mol) of 3-methylpyridine and 125 g of acetonitrile were added to a single-necked flask No. 1 to prepare a solution for later use. 14.7 g (0.123 mol) of thionyl chloride, 50 g of acetonitrile and 3.0 g of DMF were added to a single-necked flask No. 2 to prepare a solution for later use. The rest of the operations were the same as Example 3-1 to obtain 24.0 g of product KA, with a yield of 96.5% and an HPLC purity of 97.5%.

### Example 3-5

25.0 g (0.0821 mol) of KA-1 and 125 g of acetonitrile were added to a single-necked flask No. 1 to prepare a solution for later use. 14.7 g (0.123 mol) of thionyl chloride, 50 g of acetonitrile and 3.0 g of DMF were added to a single-necked flask No. 2 to prepare a solution for later use. The rest of the operations were the same as Example 3-1 to obtain 23.9 g of product KA, with a yield of 96.4% and an HPLC purity of 97.3%.

### Example 3-6

25.0 g (0.0821 mol) of KA-1, 1.5 g (0.0164 mol) of 3-methylpyridine and 125 g of acetonitrile were added to a single-necked flask No. 1 to prepare a solution for later use. 14.7 g (0.123 mol) of thionyl chloride and 50 g of acetonitrile were added to a single-necked flask No. 2 to prepare a solution for later use. The rest of the operations were the same as Example 3-1 to obtain 23.8 g of product KA, with a yield of 96.1% and an HPLC purity of 95.8%.

### Example 3-7

25.0 g (0.0821 mol) of KA-1, 1.3 g (0.0164 mol) of pyridine and 125 g of acetonitrile were added to a single-necked flask No. 1 to prepare a solution for later use. 14.7 g (0.123 mol) of thionyl chloride and 50 g of acetonitrile were added to a single-necked flask No. 2 to prepare a solution for later use. The rest of the operations were the same as Example 3-1 to obtain 23.7 g of product KA, with a yield of 95.6% and an HPLC purity of 95.6%.

### Example 3-8

25.0 g (0.0821 mol) of KA-1, 1.5 g (0.0164 mol) of 3-methylpyridine and 125 g of acetonitrile were added to a single-necked flask No. 1 to prepare a solution for later use. 14.7 g (0.108 mol) of phosphorus trichloride and 75 g of acetonitrile were added to a single-necked flask No. 2 to prepare a solution for later use. The rest of the operations were the same as Example 3-1 to obtain 24.1 g of viscous liquid, with an HPLC purity of 86.8%.

### Example 3-9

25.0 g (0.0821 mol) of KA-1, 1.5 g (0.0164 mol) of 3-methylpyridine and 125 g of acetonitrile were added to a single-necked flask No. 1 to prepare a solution for later use. 22.5 g (0.108 mol) of phosphorus pentachloride and 100 g of acetonitrile were added to a single-necked flask No. 2 to prepare a solution for later use. The rest of the operations were the same as Example 3-1 to obtain 25.1 g of viscous liquid, with an HPLC purity of 82.8%.

### Example 3-10

According to the method of Example 3-1, only thionyl chloride was replaced with oxalyl chloride to obtain 23.4 g of product KA with a yield of 94.2% and an HPLC purity of 94.7%.

### Example 3-11

According to the method of Example 3-1, only thionyl chloride was replaced with 14.6 g (0.0493 mol) of triphosgene to obtain 23.1 g of product KA with a yield of 93.2% and an HPLC purity of 94.0%.

### Example 3-12

25.0 g (0.0821 mol) of KA-1 and 125 g of acetonitrile were added to a single-necked flask No. 1 to prepare a solution for later use. 25.6 g (0.123 mol) of thionyl bromide and 50 g of acetonitrile were added to a single-necked flask No. 2 to prepare a solution for later use.

First, nitrogen gas was introduced into a four-necked flask with 25 g of acetonitrile for about 5 minutes, then chlorine gas was introduced, stirred at 10° C., then the prepared KA-1 solution in the single-necked flask No. 1 was added using a propel pump No. 1, and the prepared thionyl bromide solution in the single-necked flask No. 2 was added using a propel pump No. 2. After the introduction of KA-1 and thionyl bromide was completed, the reaction system was continuously stirred at 10° C. After HPLC detection indicated the disappearance of the raw materials, the reaction was terminated. 20% sodium hydroxide solution was added to adjust the pH to 2 to 3, the phases were separated, and then ethyl acetate was added for extraction. The organic phases were combined and rotary evaporated to obtain 23.5 g of product KA, with a yield of 94.6% and an HPLC purity of 97.1%.

### Example 3-13

25.0 g (0.0821 mol) of KA-1 and 125 g of acetonitrile were added to a single-necked flask No. 1 to prepare a solution for later use. 25.6 g (0.123 mol) of thionyl bromide and 50 g of acetonitrile were added to a single-necked flask No. 2 to prepare a solution for later use, and 15.7 g of bromine and 50 g of acetonitrile were added to a single-necked flask No. 3 to prepare a solution for later use.

Nitrogen gas was introduced into a four-necked flask with 25 g of acetonitrile, then the prepared KA-1 solution in the single-necked flask No. 1 was added using a propel pump No. 1, the prepared thionyl bromide solution in the single-necked flask No. 2 was added using a propel pump No. 2, and the prepared bromine solution in the single-necked flask No. 3 was added using a propel pump No. 3. The three streams of the materials were maintained at constant rate and their introduction was terminated at the same time. The temperature during the introduction was 5=2° C. After the introduction was completed, the reaction system was continuously stirred at 5° C. After HPLC detection indicated the disappearance of the raw materials, the reaction was terminated. 20% sodium hydroxide solution was added to adjust the pH to 2 to 3, the phases were separated, and then ethyl acetate was added for extraction. The organic phases were combined and rotary evaporated to obtain 23.8 g of product KA, with a yield of 94.4% and an HPLC purity of 93.2%.

### Example 4-1

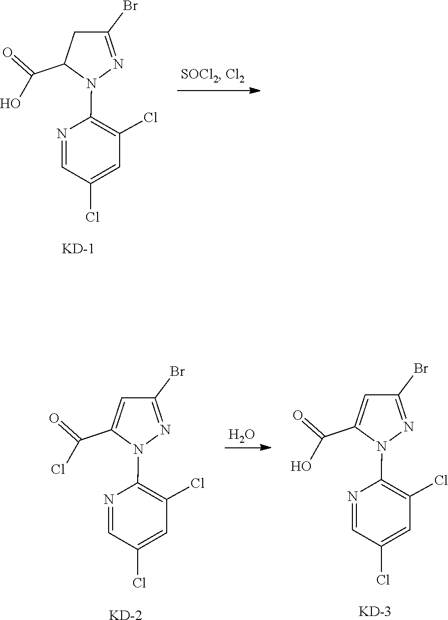

25.0 g (0.0738 mol) of KD-1 and 125 g of acetonitrile were added to a single-necked flask No. 1 to prepare a solution for later use. 13.2 g (0.111 mol) of thionyl chloride and 50 g of acetonitrile were added to a single-necked flask No. 2 to prepare a solution for later use.

First, nitrogen gas was introduced into a four-necked flask with 25 g acetonitrile for about 5 minutes, then chlorine gas was introduced, stirred at 15° C., then the KD-1 solution prepared in the single-necked flask No. 1 was slowly added using a propel pump No. 1, and the thionyl chloride solution prepared in the single-necked flask No. 2 was slowly added using a propel pump No. 2. After the introduction of KD-1 and thionyl chloride was completed, the reaction system was heated to 30° C. and stirred. After HPLC detection indicated the disappearance of the raw materials, the reaction was terminated. 20% sodium hydroxide solution was added to adjust the pH to 2 to 3, the phases were separated, and then ethyl acetate was added for extraction. The organic phases were combined and rotary evaporated to obtain 23.0 g of product KD-3, with a yield of 92.3% and an HPLC purity of 91.8%.

### Example 4-2

25.0 g (0.0738 mol) of KD-1 and 125 g of dioxane were added to a single-necked flask No. 1 to prepare a solution for later use. 13.2 g (0.111 mol) of thionyl chloride and 50 g of dioxane were added to a single-necked flask No. 2 to prepare a solution for later use. The rest of the operations were the same as Example 4-1 to obtain 23.4 g of product KD-3, with a yield of 94.0% and an HPLC purity of 91.2%.

### Example 4-3

25.0 g (0.0738 mol) of KD-1 and 125 g of tetrahydrofuran were added to a single-necked flask No. 1 to prepare a solution for later use. 13.2 g (0.111 mol) of thionyl chloride and 50 g of tetrahydrofuran were added to a single-necked flask No. 2 to prepare a solution for later use. The rest of the operations were the same as Example 4-1 to obtain 23.0 g of product KD-3, with a yield of 92.3% and an HPLC purity of 91.8%.

### Example 4-4

25.0 g (0.0738 mol) of KD-1 and 125 g of tetrahydrofuran were added to a single-necked flask No. 1 to prepare a solution for later use. 13.2 g (0.111 mol) of thionyl chloride and 50 g of tetrahydrofuran were added to a single-necked flask No. 2 to prepare a solution for later use.

First, nitrogen gas was introduced into a four-necked flask with 25 g of tetrahydrofuran for about 5 minutes, and then chlorine gas was introduced, stirred at 50° C., and then the prepared KD-1 solution in the single-necked flask No. 1 was slowly added using a propel pump No. 1, and the prepared thionyl chloride solution in single-necked flask No. 2 was slowly added using a propel pump No. 2. After the introduction of KD-1 and thionyl chloride was completed, the reaction system was heated to 50° C. and stirred. After HPLC detection indicated the disappearance of the raw materials, the reaction was terminated. 20% sodium hydroxide solution was added to adjust the pH to 2 to 3, the phases were separated, and then ethyl acetate was added for extraction. The organic phases were combined and rotary evaporated to obtain 23.7 g of product KD-3, with a yield of 95.2% and an HPLC purity of 95.8%.

### Example 5

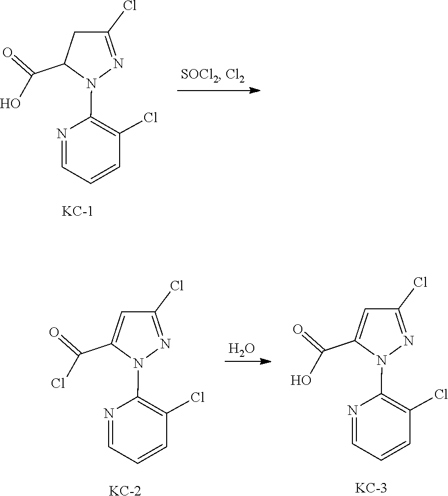

30.0 g (0.115 mol) of KC-1 and 120 g of acetonitrile were added to a single-necked flask No. 1 to prepare a solution for later use. 20.6 g (0.173 mol) of thionyl chloride and 60 g of acetonitrile were added to a single-necked flask No. 2 to prepare a solution for later use.

First, nitrogen gas was introduced into a four-necked flask with 25 g of acetonitrile for about 5 minutes, then chlorine gas was introduced, stirred at 5° C., and then the KC-1 solution prepared in the single-necked flask No. 1 was slowly added using a propel pump No. 1, and the thionyl chloride solution prepared in the single-necked flask No. 2 was slowly added using a propel pump No. 2. After the introduction of KC-1 and thionyl chloride was completed, the reaction system was heated to 15° C. and stirred. After HPLC detection indicated the disappearance of the raw materials, the reaction was terminated. 20% sodium hydroxide solution was added to adjust the pH to 2 to 3, the phases were separated, and then ethyl acetate was added for extraction. The organic phases were combined and rotary evaporated to obtain 27.9 g of product KC-3, with a yield of 94.1% and an HPLC purity of 94.3%.

### Example 6

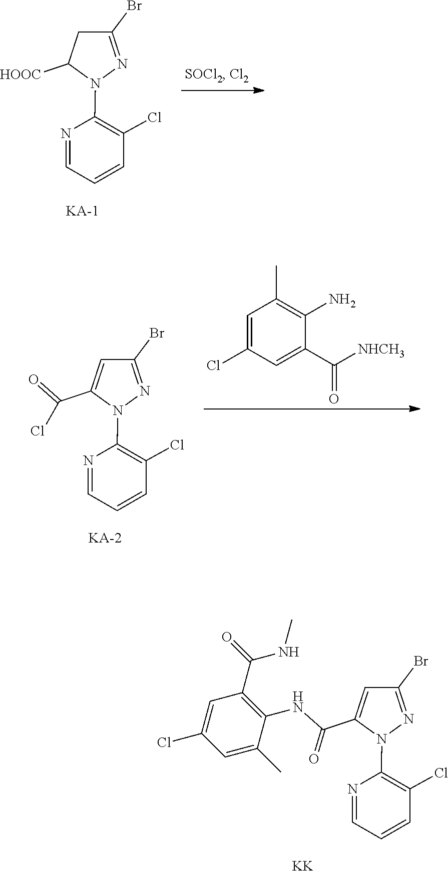

25.0 g (0.0821 mol) of KA-1 and 125 g of acetonitrile were added to a single-necked flask No. 1 to prepare a solution for later use. 14.7 g (0.123 mol) of thionyl chloride and 50 g of acetonitrile were added to a single-necked flask No. 2 to prepare a solution for later use.

First, nitrogen gas was introduced into a four-necked flask with 25 g acetonitrile for about 5 minutes, then chlorine gas was slowly introduced, stirred at 15° C., then the KA-1 solution prepared in the single-necked flask No. 1 was slowly added using a propel pump No. 1, and the thionyl chloride solution prepared in the single-necked flask No. 2 was slowly added using a propel pump No. 2. After the introduction of KA-1 and thionyl chloride was completed, the reaction system was heated to 30° C. and stirred. After HPLC detection indicated the disappearance of the raw materials, the reaction was terminated.

The reaction solution was subjected to distillation under reduced pressure at a distillation temperature not exceeding 45° C. to obtain 185 g of a mixed liquid. 16.3 g of 2-amino-5-chloro-N,3-dimethylbenzamide was added to the mixed liquid in batches, stirred at 15° C. for about 0.5 hours, then heated to 40° C. and stirred for 2 hours. After HPLC detection showed that about 8% of KA-2 was not completely reacted. Most of hydrogen chloride was removed by vacuum distillation, and then 1.6 g (0.0164 mol) of 3-methylpyridine and 0.9 g (0.00821 mol) of methylsulfonyl chloride were added and stirred at 15° C. After HPLC detection indicated the disappearance of the raw materials, the reaction was terminated. 75 g of water was slowly added dropwise, stirred at room temperature for crystallization. After filtration, the filter cake was dried to obtain 36.5 g of off-white solid product KK, with a yield of 92.1%.

### Example 6-1

25.0 g (0.0821 mol) of KA-1 and 125 g of acetonitrile were added to a single-necked flask No. 1 to prepare a solution for later use. 14.7 g (0.123 mol) of thionyl chloride and 50 g of acetonitrile were added to a single-necked flask No. 2 to prepare a solution for later use.

First, nitrogen gas was introduced into a four-necked flask with 25 g of acetonitrile for about 5 minutes, then chlorine gas was slowly introduced, stirred at 30° C., then the KA-1 solution prepared in the single-necked flask No. 1 was slowly added using a propel pump No. 1, and the thionyl chloride solution prepared in the single-necked flask No. 2 was slowly added using a propel pump No. 2. After the introduction of KA-1 and thionyl chloride was completed, the reaction system was heated to 30° C. and stirred. After HPLC detection indicated the disappearance of the raw materials, the reaction was terminated.

The reaction solution was subjected to distillation under reduced pressure at a distillation temperature not exceeding 45° C. to obtain 185 g of a mixed liquid. 16.3 g of 2-amino-5-chloro-N,3-dimethylbenzamide, 32.6 g of acetonitrile and 16.8 g of 3-methylpyridine were added to another three-necked flask, stirred at 20° C. for 10 minutes, then 185 g of the mixed liquid prepared above was slowly added dropwise into the three-necked flask, the internal temperature was kept at 20 to 25° C. for reaction. After the dropwise addition was completed, the temperature was maintained to continue the reaction. After HPLC detection indicated that the reaction was completed, 68.6 g of 7.2% dilute hydrochloric acid was added dropwise into the three-necked flask, stirred at 20° C. for 90 minutes. After filtration, the filter cake was dried to obtain 37.0 g of off-white solid, with a yield of 93.3%.

### Example 7

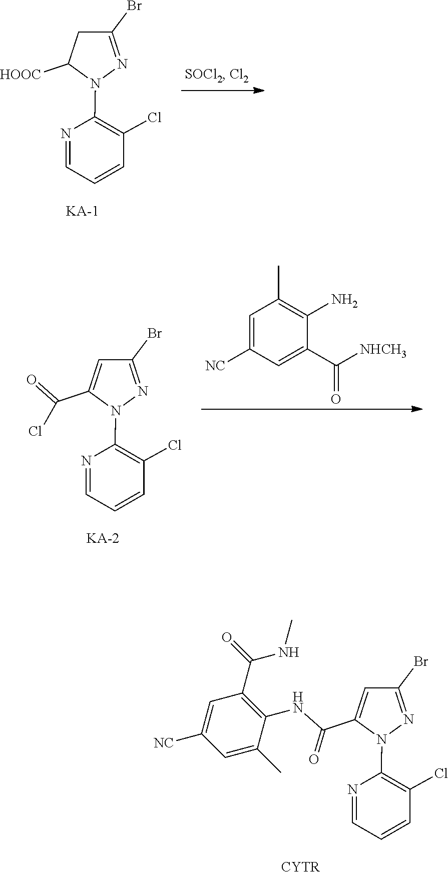

25.0 g (0.0821 mol) of KA-1 and 125 g of acetonitrile were added to a single-necked flask No. 1 to prepare a solution for later use. 14.7 g (0.123 mol) of thionyl chloride and 50 g of acetonitrile were added to a single-necked flask No. 2 to prepare a solution for later use.

First, nitrogen gas was introduced into a four-necked flask with 25 g acetonitrile for about 5 minutes, then chlorine gas was introduced, stirred at 15° C., then the KA-1 solution prepared in the single-necked flask No. 1 was slowly added using propel a pump No. 1, and the thionyl chloride solution prepared in the single-necked flask No. 2 was slowly added using propel pump No. 2. After the introduction of KA-1 and thionyl chloride was completed, the reaction was heated to 30° C. and stirred. After HPLC detection indicated the disappearance of the raw materials, the reaction was terminated.

The reaction solution was subjected to distillation under reduced pressure at a distillation temperature not exceeding 45° C. to obtain 185 g of a mixed liquid. 15.5 g of 2-amino-5-cyano-N,3-dimethylbenzamide was added to the mixed liquid in batches, stirred at 15° C. for about 0.5 hours, then heated to 40° C. and stirred for 2 hours. After HPLC detection indicated that about 6% of KA-2 was not completely reacted, most of hydrogen chloride was removed by vacuum distillation, and then 1.6 g (0.0164 mol) of 3-methylpyridine and 0.9 g (0.00821 mol) of methylsulfonyl chloride were added and stirred at 15° C. After HPLC detection indicated the disappearance of the raw materials, the reaction was terminated. 100 g of water was slowly added dropwise, and stirred at room temperature for crystallization. After filtration, the filter cake was dried to obtain 36.3 g of off-white solid product CYTR, with a yield of 93.4%.

### Example 8: Preparation of Tetrachlorantraniliprole

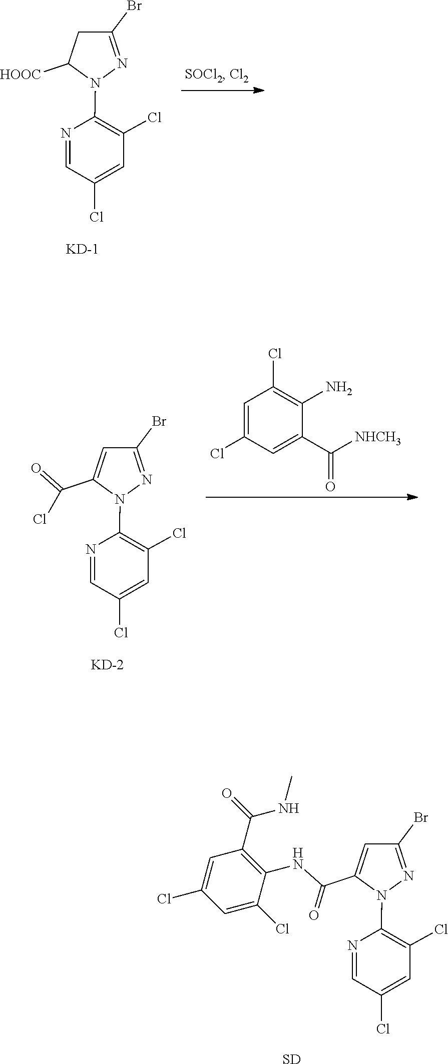

25.0 g (0.0738 mol) of KD-1, 125 g of acetonitrile and 1.5 g (0.0164 mol) of 3-methylpyridine were added to a single-necked flask No. 1 to prepare a solution for later use. 13.2 g (0.111 mol) of thionyl chloride and 50 g of acetonitrile were added to a single-necked flask No. 2 to prepare a solution for later use.

First, nitrogen gas was introduced into a four-necked flask with 25 g of acetonitrile for about 5 minutes, then chlorine gas was introduced, stirred at 15° C., then the prepared KA-1 solution in the single-necked flask No. 1 was slowly added using a propel pump No. 1, and the prepared thionyl chloride solution in the single-necked flask No. 2 was slowly added using a propel pump No. 2. After the introduction of KA-1 and thionyl chloride was completed, the reaction system was heated to 30° C. and stirred. After HPLC detection indicated the disappearance of the raw material, the reaction was terminated.

The reaction solution was subjected to distillation under reduced pressure at a distillation temperature not exceeding 45° C. to obtain 190 g of a mixed liquid. 15.4 g (0.0701 mol) of 2-amino-3,5-dichlorobenzamide was added to the mixed liquid in batches, stirred at 15° C. for about 0.5 hours, then heated to 40° C. and stirred for 2 hours. After HPLC detection indicated that about 5% of KA-2 was not completely reacted, most of hydrogen chloride was removed by vacuum distillation, and then 1.6 g (0.0164 mol) of 3-methylpyridine and 0.9 g (0.00821 mol) of methylsulfonyl chloride were added and stirred at 15° C. After HPLC detection indicated the disappearance of the raw material, the reaction was terminated. 100 g of water was slowly added dropwise, and stirred for crystallization at room temperature. After filtration, the filter cake was dried to obtain 34.0 g of solid product SD with a yield of 85.7%.

### Comparative Example 1

25.0 g (0.0821 mol) of KA-1 and 125 g of acetonitrile were added to a single-necked flask No. 1 to prepare a solution for later use. 14.7 g (0.123 mol) of thionyl chloride and 50 g of acetonitrile were added to a single-necked flask No. 2 to prepare a solution for later use.

First, nitrogen gas was introduced into a four-necked bottle with 25 g of acetonitrile for about 5 minutes, and stirred at 15° C. Then, the KA-1 solution prepared in the single-necked bottle No. 1 was slowly added using a propel pump No. 1, and the thionyl chloride solution prepared in the single-necked bottle No. 2 was slowly added using a propel pump No. 2. After the introduction of KA-1 and thionyl chloride was completed, the reaction system was slowly heated to 30° C. and stirred. After HPLC detection indicated the disappearance of the raw materials, the reaction was terminated. 20% sodium hydroxide solution was added to adjust the pH to 2 to 3, phases were separated, and then ethyl acetate was added for extraction. The organic phases were combined and rotary evaporated to obtain 23.7 g of residue. HPLC showed that the product KA accounted for only 49% (with a yield of 46%), the raw material KA-1 remained 28%, and the rest was impurities.

### Comparative Example 2

25.0 g (0.0821 mol) of KA-1, 0.2 g of N,N-dimethylformamide and 125 g of dichloromethane were added to a three-necked flask. 24.4 g (0.205 mol) of thionyl chloride and 24 g of dichloromethane were added to a dropping funnel, then slowly added dropwise into the reaction flask. The temperature was controlled at 35 to 40° C. and reaction was carried out for 2 to 3 hours. HPLC showed that the product KA accounted for 77%, and the rest was impurities.

Although the specific embodiments of the present invention have been described in detail, those skilled in the art should understand that various modifications and substitutions can be made to those details based on all the teachings disclosed, and these changes are within the scope of protection of the present invention. The full scope of the present invention is given by the appended claims and any equivalents thereof.

1. A method for preparing a compound represented by Formula I, comprising the following steps:
reacting a carboxylic acid represented by Formula I-1 with a halogenating agent and an elemental halogen X₂ to obtain the compound represented by Formula I: 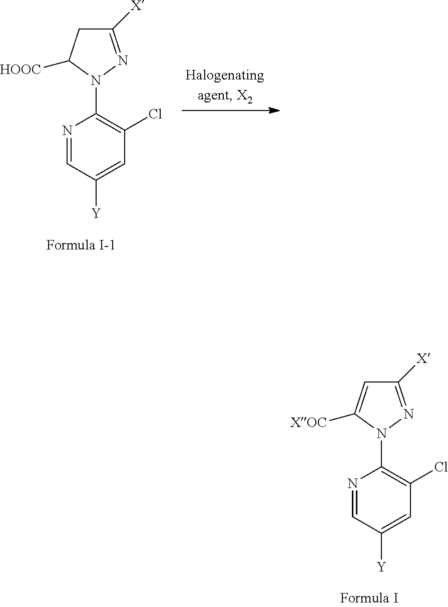
in Formula I and Formula I-1, X is halogen; X′ is halogen, C₁₋₆ alkyl, C₁₋₆ alkoxy or C₁₋₆ haloalkoxy; X″ is a halogen; Y is H or halogen; X, X′ and X″ are the same or different.

2. The method according to claim 1, wherein the halogenating agent is a chlorinating agent or a brominating agent.

3. The method according to claim 1, wherein the molar ratio of the carboxylic acid to the halogenating agent is 1:(1.0-3.0), for a polymer such as triphosgene, the calculation is based on its monomer unit.

4. The method according to claim 1, wherein the elemental halogen is chlorine or bromine.

5. The method according to claim 1, wherein the halogenating agent is thionyl chloride and the elemental halogen is chlorine.

6. The method according to claim 1, wherein the reaction is carried out in a solvent; preferably, the solvent is selected from the group consisting of acetonitrile, chlorobenzene, dioxane, tetrahydrofuran, dichloromethane, dichloroethane, chloroform and carbon tetrachloride; preferably, the solvent is acetonitrile.

7. The method according to claim 6, comprising the following steps:
adding the carboxylic acid, halogenating agent (e.g., thionyl chloride) and elemental halogen (e.g., chlorine) to the solvent, respectively, to obtain solution containing the carboxylic acid, halogenating agent or elemental halogen;
adding (e.g., adding simultaneously) the solution containing the carboxylic acid and the solution containing the halogenating agent obtained above to the solution containing the elemental halogen, and reacting to obtain the compound represented by Formula I.

8. The method according to claim 1, wherein the carboxylic acid reacts with the halogenating agent and the elemental halogen in the presence of an acid-binding agent;
preferably, the acid-binding agent is one or more selected from the group consisting of triethylamine, pyridine, 3-methylpyridine and 2-methylpyridine.

9. The method according to claim 1, wherein the carboxylic acid reacts with the halogenating agent and the elemental halogen in the presence of a catalyst;
preferably, the catalyst is one or more selected from the group consisting of N,N-dimethylformamide, N,N-dimethylacetamide, pyridine and triethylamine.

10. A method for preparing a compound represented by Formula II, comprising the following steps:
using the Formula I-1 as a raw material to obtain the compound represented by Formula I according to the steps as defined in the method according to claim 1;
hydrolyzing the compound represented by Formula I to obtain the compound represented by Formula II, 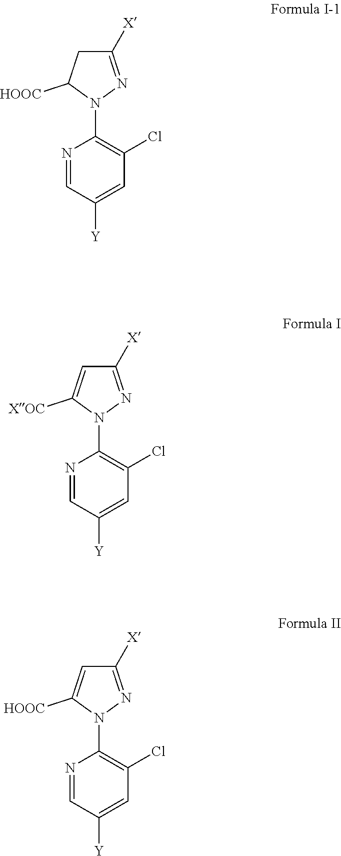
wherein, the groups in Formula I, I-1 and II are as defined in claim 1.

11. A method for preparing a compound represented by Formula III, comprising the following steps:
reacting a carboxylic acid represented by Formula I-1 with a halogenating agent and a elemental halogen according to the method according to claim 1;
further reacting the product obtained in the previous step with a compound represented by Formula IV to obtain a compound represented by Formula III; 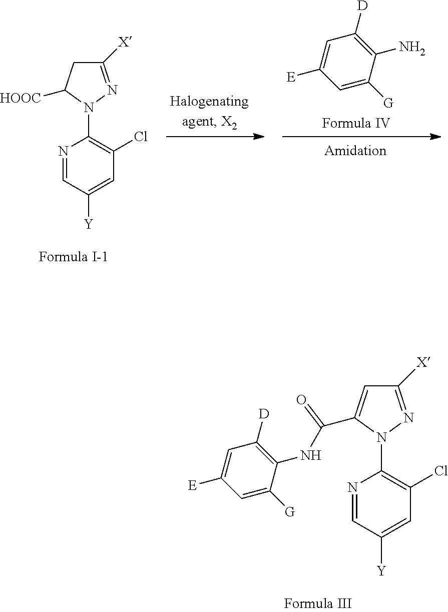
wherein, D is halogen or C1-6 alkyl; E is halogen or cyano; G is —C(O)NH—C1-6 alkyl, —C(S)NH—C1-6 alkyl, —C(O)NH—C1-6 alkylene-C3-6 cycloalkyl, —C(S)NH—C1-6 alkylene-C3-6 cycloalkyl, or 5- to 6-membered heteroaryl unsubstituted or substituted with halogen or C1-6 alkyl; the remaining groups are as defined in claim 1;
preferably, D is Cl, Br or C₁₋₂ alkyl; E is Cl, Br or cyano; G is —C(O)NH—C1-4 alkyl, —C(S)NH—C1-4 alkyl, —C(O)NH—C1-2 alkylene-C3-6 cycloalkyl, —C(S)NH—C1-2 alkylene-C3-6 cycloalkyl, or 5-membered heteroaryl unsubstituted or substituted with halogen or CC1-2 alkyl;
preferably, D is Cl, Br or methyl; E is Cl or cyano; G is selected from

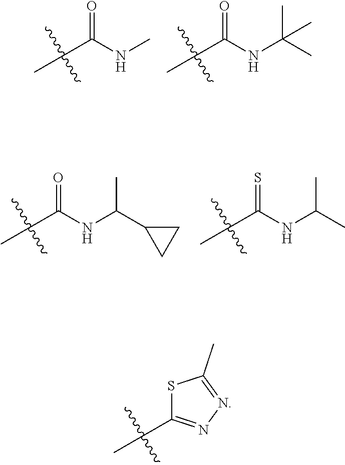

12. The method according to claim 11, wherein the compound represented by Formula III is a bisamide insecticide;
preferably, the compound represented by Formula III is an ortho-amido benzamide insecticide;
preferably, the ortho-amido benzamide insecticide is selected from the group consisting of chlorantraniliprole, cyantraniliprole, tetrachlorantraniliprole, cyclaniliprole, tetraniliprole, tiorantraniliprole, Fuyangchongxian'an (ZJ3757).

13. (canceled)

14. The method according to claim 1, wherein X is Cl or Br; X′ is Cl, Br, C₁₋₂ alkyl, C₁₋₂ alkoxy or C₁₋₂ haloalkoxy; X″ is Cl or Br; Y is H, Cl or Br; X, X′ and X″ are the same or different.

15. The method according to claim 1, wherein X is Cl; X′ is Cl or Br; X″ is Cl or Br; Y is H or Cl; X, X′ and X″ are the same or different.

16. The method according to claim 1, wherein X is Cl; X′ is Br; X″ is Cl; Y is H.

17. The method according to claim 2, wherein the chlorinating agent is selected from the group consisting of sulfinyl chlorides (e.g., thionyl chloride), sulfonyl chlorides (e.g., methanesulfonyl chloride or trichloromethanesulfonyl chloride), acyl chlorides (e.g., methoxyacetyl chloride, oxalyl chloride, phosgene or triphosgene) and phosphorus chlorides (e.g., phosphorus trichloride or phosphorus pentachloride).

18. The method according to claim 2, wherein the brominating agent is selected from the group consisting of thionyl bromide and phosphorus tribromide.

19. The method according to claim 3, wherein the molar ratio of the carboxylic acid to the halogenating agent is 1:(1.0-2.0) such as 1:(1.0-1.5), for a polymer such as triphosgene, the calculation is based on its monomer unit.

20. The method according to claim 7, wherein the temperature of the reaction system during the addition process is controlled; preferably, the temperature of the reaction system during the addition process is controlled to be 5 to 70° C., preferably 5 to 20° C., for example, 10 to 15° C.

21. The method according to claim 7, wherein the reaction is carried out at −10 to 80° C., preferably 10 to 50° C., more preferably 10 to 45° C., for example, 10 to 40° C., 15 to 45° C. or 30 to 45° C.
